(19) United States
(12) Patent Application Publication
(10) Pub. No.: US 2024/0429964 A1
(43) Pub. Date: Dec. 26, 2024

(54) COMMUNICATION APPARATUS FOR USE WITH ELECTRONIC COMMUNICATION ELEMENT, ELECTRONIC COMMUNICATION ELEMENT AND USES THEREOF

(71) Applicant: Ares Trading S.A., Aubonne (CH)

(72) Inventors: Alessandro Macor; Wim Saelen, Coinsins (CH); Charles Henderson, Basel (CH); Peter Relph, Basel (CH)

(21) Appl. No.: 18/676,669

(22) Filed: May 29, 2024

Related U.S. Application Data
(63) Continuation of application No. 17/415,906, filed on Jun. 18, 2021, now abandoned.

(51) Int. Cl. H04B 1/707; G06K 7/00; G06K 19/07; G06K 19/077; H04B 5/77

(52) U.S. Cl. CPC H04B 1/707; G06K 7/0008; G06K 19/0716; G06K 19/0723; G06K 19/07749; H04B 5/77; G06K 19/0702

(57) ABSTRACT

There is described a communication system (1) comprising a central unit (3) and a communication element (20). The central unit (3) comprises at least one RF antenna (4), a transmitter (6) configured to transmit a RF signal and a receiver (8) configured to receive a backscattered RF signal. The communication element (20) comprises a RF antenna (22) and a modulator (23) configured to backscatter the RF signal. The communication element (20) comprises a means for modulating the backscattered RF signal into a spread spectrum backscattered RF signal, and a means for supplying power.

### FIELD OF THE INVENTION

The present invention relates to a communication system including an electronic communication element, such as for example a label or a tag, based on backscatter technology. More specifically, it relates to a system and communication elements, such as for example labels or tags, based on radio frequency identification (RFID) technology attached to consumables for use in identifying, recognizing, and registering such consumables in a home environment.

### BACKGROUND

The connection of low cost, disposable, devices to the cloud is of significant interest. For example, by connecting drug packaging or drug delivery devices such as auto-injectors, remotely monitoring patient adherence in an in-home environment, and actions taken to ultimately improve desired patient outcome. To enable many of these applications, the communication solution needs to work seamlessly, without the need for re-occurring additional actions on the part of the patient.

RFID tag technology is an attractive technology to enable such systems, because the tag hardware is simple, and consequently low cost and easily integrated into packaging or labels. Compared to other two-way radio communications systems, the simplicity is enabled by using the incident RF signal transmitted from the ‘reader’ to both power the tag; and the tag transmitting it's data by backscatter modulation of this incident signal. This removes the need to include a full radio transmitter and power source in the tag, as compared to conventional two-way radio systems.

Applications in which sensors and interfaces are added to RFID tags are also starting to evolve to specifically enable device monitoring. However, it's application is limited due to the short range between an RFID reader and tag. This requires the user to take a specific action to ‘read’ the device, by going in proximity to a reader, and thus the system is not seamless and fulfills one of the needs as described above.

Examples of widely used approaches include EPC Gen2 tags operating in the UHF band, which typically have a range <10 m within one direction of the reader, and HF RFID or NFC devices which operate using near field coupling with ranges <1 m depending upon antenna size.

The conventional UHF RFID system is well-known and typically comprises:
a) a reader that includes at least one RF antenna, a transmitter configured to transmit a RF signal, a receiver configured to receive a backscattered RF signal, and processing system to control operation and manage the protocol.
b) a tag (or communication element) that includes a RF antenna, energy harvesting circuit to power the tag from the incident RF, receiver to decode data on the RF signal that is sent by the reader, a modulator configured to backscatter the RF signal received from the central unit and thus send data, and processing logic to handle the protocol.

However, the range of conventional UHF RFID tags is limited by several features of their communication system. These include:
i. that the transmitted power from the hub/reader is limited to a maximum, by law, and most readers use this maximum power. Therefore, the communication range cannot be increased further by increasing the transmitted power;
ii. that powering of the communication element (tag or label) from the RF transmitted by the hub/reader means that a minimum amount of energy needs to be present at the communication element in order for it to operate. The amount of power that can be received from the hub/reader signal decreases with distance from the hub/reader. Therefore, there is a maximum operable range; and
iii. that the sensitivity at the hub/reader is sufficient to accurately receive the backscattered signal from the communication element (tag or label). A particular challenge here is that the backscattered signal that the hub/reader needs to receive is significantly weaker than the outward signal that is transmitted to the communication element (tag or label) while both the outward signal and the backscattered signal are at the same frequency. As a consequence, the transmitter ‘deafens’ the receiver.

Thus, although conventional RFID tags work well in particular applications and are economical to be incorporated in consumables, the operational range of such conventional RFID tag systems is too limited in order to cover an entire home with a non-mobile reader. In addition, the RFID information of each such tag is static and cannot change over time and therefore any parameter of the corresponding product (such as for example operational status) cannot be dynamically included in the conventional passive RFID tag.

In view of all the above, there is a need for improved communication systems and communication elements based on backscatter technology that not only can dynamically specify changing information, such as operational status, regarding its associated product, but importantly extend the operation range of conventional RFID tag and reader systems while maintaining such communication system as economical for use with tracking consumables in a home environment and at the time same simplify the use, for example through a seamless experience for the user to connect and have the consumables communicate with a communication system.

### BRIEF SUMMARY OF THE INVENTION

Described herein is a backscatter-based communications system with features to increase the operational range in order to provide a solution to the aforementioned drawbacks of conventional RFID technology and methods of using such communication system in identifying, recognizing, and registering consumables in a home environment. Such communication system as described herein comprises a central unit (transmitter/reader) and one or more communication elements.

In one aspect the current invention describes a system that like conventional UHF RFID tags is simple through the use of backscatter communications but has an increased range such that a single reader (hereafter referred to as ‘hub’ or ‘central unit’) can communicate with communication elements, associated with consumables, within the size of a typical home. In addition, the current invention enables a more seamless user experience.

In an embodiment a modified RFID communications system, for example a modified UHF RFID communications system, is described in which:
The communication element is powered from an energy source which is substantially independent of the RF signal sent from the hub.
The sensitivity of the hub or central unit to the signal from the communication element is increased through a combination of two or more features that include:
   Dual antennas at the hub or central unit to minimize crosstalk between the transmit and receive path.
   Carrier self-cancellation techniques in the hub or central unit, to actively cancel crosstalk between the transmit and receive paths.
   Binary phase shift keying at the communication element, to enable maximum power in the backscattered signal, as compared to amplitude modulation.
   Direct sequence spread spectrum modulation at the communication element, and corresponding de-spreading at the hub or central unit. This increases the bandwidth of the backscattered signal from the communication element, spectrally separating it from the carrier transmitted by the hub or central unit, thus enabling it to be more easily separated and decoded by the hub or central unit.
   Use of a low phase noise signal source at the hub central unit, to minimize the bandwidth of the transmitted carrier from the hub or central unit, thus enabling the benefits of the spreading of the backscattered signal to be maximized.
   Use of a very low bandwidth communications data rate, thereby increasing receiver sensitivity and hence range.

Objects of the invention are achieved by the communication system (including a communication element and hub or central unit as described and claimed herein) according to the claims.

In order to further enable the communication system for a seamless connectivity solution to sensors on packaging, the communication system in further embodiments includes the following additional features:
An Isotropic antenna at the hub or central unit, to enable it to cover a whole home from any position.
On board processing and memory on the communication element to enable intelligent applications.
Addition of sensors, for example a humidity sensor, a temperature sensor, integrity sensor, and a light intensity sensor, operably connected to the communication element.
Addition of a user interface, for example consisting of a button, LED and/or display.

Furthermore, in order to enable reduced costs and small form factor at the consumable, some or all of the components in the communication element can be fabricated using printed electronics technologies. For example, to create a communication element that takes the form of a label.

Further, disclosed herein, according to an aspect of the invention, is a communication system comprising: a) a central unit comprising at least one RF antenna, a transmitter configured to transmit a RF signal and a receiver configured to receive a backscattered signal; and b) a communication element comprising a RF antenna and a modulator configured to backscatter the RF signal received from the central unit. The communication element comprises a modulator for modulating the backscattered RF signal to obtain a spread spectrum backscattered RF signal and includes a means for supplying power to the modulator.

In an advantageous embodiment the means for supplying power to the communication element is independent from the energy of the RF signal transmitted from the hub or central unit to the communication element.

In an embodiment, the transmitter further comprises a low phase noise signal source.

In an embodiment, the central unit further comprises a means for carrier cancellation self-interference mitigation selected from the presence of at least two RF antennas at the central unit, analog carrier cancellation, digital carrier cancellation, and the use of a high pass filter.

In an embodiment, the spread spectrum backscattered RF signal is a direct sequence spread spectrum backscattered RF signal.

In an embodiment, the central unit comprises means for de-spreading the spread spectrum backscattered RF signal.

In an embodiment, the de-spreading means uses a very low data bandwidth rate.

In an embodiment, the RF antenna is an isotropic antenna.

In an embodiment, the maximum operable range between the central unit and the communication element is at least up to 50 m, advantageously at least up to 65 m, more advantageously at least up to 100.

In an embodiment, the communication element does not contain a precision frequency reference, for example a crystal oscillator.

In an embodiment, the communication element further comprises a processing unit.

In an embodiment, the communication element further comprises a memory means.

In an embodiment, the communication element comprises a memory means selected from a printed memory and silicon semiconductor-based memory, for example a memory chip or an integrated memory on the communication element.

In an embodiment, the communication element comprises printed electronics.

In an embodiment, the means for supplying power in the communication element comprises a single battery or an energy harvesting means.

In an embodiment, the single battery is a printed battery.

In an embodiment, the communication element comprises a means for Binary Phase Shift Keying or Binary Amplitude Shift Keying of the RF signal.

In an advantageous embodiment, the communication element is configured for two-way communication between itself and the central unit.

In an embodiment, the communication element further comprises one or more sensors selected from the group consisting of a humidity sensor, a temperature sensor, integrity sensor, and a light intensity sensor, operably connected to the communication element, optionally such sensors are integrated into the communication element.

In an embodiment, the communication system further comprises a user interface, which optionally is connected or integrated with the communication element.

In an embodiment, the user interface is selected from a button, LED, and display. Objects of the invention are achieved by the communication element according to claims.

Disclosed herein, according to another aspect of the invention, is a communication element comprising a RF antenna, a receiver configured to backscatter a RF signal, a means for supplying power and a modulator configured for modulating the backscattered RF signal to obtain a spread spectrum backscattered RF signal.

In an embodiment, the communication element further comprises a memory means.

In an embodiment, the memory means is selected from a printed memory and a silicon semiconductor-based memory, for example a memory chip or an integrated memory on the communication element.

In an embodiment, the communication element further comprises a processing unit.

In an embodiment, the communication element comprises printed electronics.

In an embodiment, the means for supplying power is a (single) battery or an energy harvesting means.

In an embodiment, the (single) battery is a printed battery.

In an embodiment, the modulator is configured for modulating the RF signal to obtain a spread spectrum backscattered RF signal is a direct sequence spread spectrum modulator.

In an embodiment, the memory means comprises a means to store and read/write data onto the communication element through a communication interface.

In an embodiment, the communication element further comprising one or more sensors selected from the group consisting of a humidity sensor, a temperature sensor, integrity sensor, and a light intensity sensor, communicatively connected to the communication element.

In an embodiment, the communication element further comprises a timer mechanism.

In an embodiment, the communication element further comprises a user interface.

In an embodiment, the user interface is selected from a button, LED, and display.

In an embodiment, the communication element is an integral part of the packaging of a consumable or a medicament. As described herein the term integral signifies that the communication element is completely or partly part of the packaging as opposed to being a separate communication element which may be attached to a packaging. For example where such communication element is an integral part of the packaging such communication element is fully or partly incorporated into such packaging or consumable or alternatively is directly printed onto the packaging.

In an embodiment the central unit or hub comprises a means for connecting to other devices or cloud services, such means can be any gateway which can establish such connection.

Also disclosed herein, according to another aspect of the invention, is a method of transmitting information between a central unit and a consumable or medicament container using the communication system as described above.

In an advantageous embodiment such method is used in identifying, recognizing, and registering consumables, for example medication containers, in a home environment

In an embodiment, the medication container is selected from a vial, a blister pack, a syringe, a cartridge, and a drug delivery device.

### BRIEF DESCRIPTION OF THE DRAWINGS

The above-mentioned and other advantages and objects of this invention will become more apparent, and the invention itself will be better understood by reference to the following description of embodiments of the invention together with the accompanying drawings, wherein:

FIG. 1 illustrates a schematic representation of the communication system according to an embodiment of the invention;

FIG. 2 illustrates a schematic view of the central unit of the communication system of FIG. 1, and

FIG. 3 is illustrates a schematic view of the communication element of the communication system of FIG. 1

### DETAILED DESCRIPTION OF THE INVENTION

Embodiments of the inventive concept are described below with reference to the accompanying drawings where appropriate. These embodiments are presented as teaching examples and should not be construed to limit the scope of the inventive concept.

The communications system according to an embodiment of the invention is particularly adapted for monitoring drug usage of patients in a home environment in order to assist them for optimal therapeutic benefits.

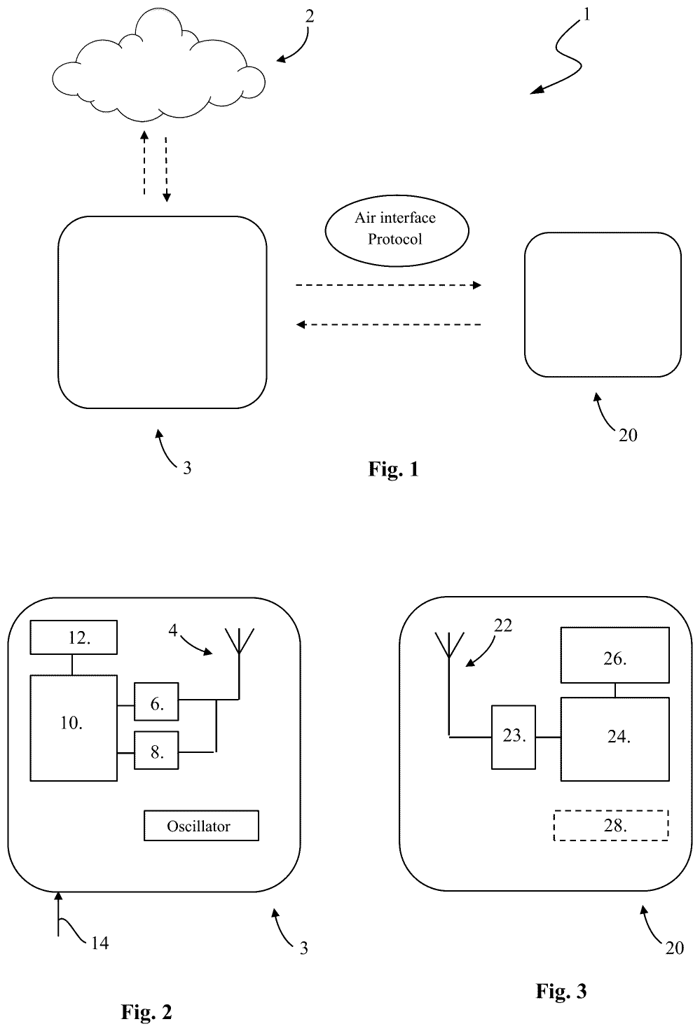

In particular, as illustrated in FIG. 1, the communication system 1 is preferably part of a cloud-based services for patients. The cloud services may be for example a healthcare provider which may provide medical feedbacks based on the information transmitted by the communication system 1. In this respect, the communications system 1 is configured to communicate with a cloud service 2 and with one or more communication elements 20. The communication element 20 may be in the form of an electronic label, tag or module operably connected to a drug container or a drug delivery device and optionally connected with one or more sensors. Such operably connected communication element can be attached to, or form an integral part of, a drug container or drug delivery device and can register any change to such drug container or drug delivery device. Such change being associated with the usage of the drug container or drug delivery device.

In an embodiment, the communication element 20 may be an integral part of a drug container and may be adhered for example to a vial or a cartridge containing a liquid drug or to a blister pack. The drug container may be provided with sensors operably connected or directly integrated into the communication element 20 in order to dynamically change information contained in the communication element 20 as a function of the drug available to the patient(s). In an embodiment, the communication element 20 may be adhered to a drug delivery device to dynamically store information in the communication element 20 based on the operating status of the drug delivery device.

Other sensors may be operably or directly integrated into the communication element 20. For example, one or more of these sensors may be selected from the group consisting of a humidity sensor, a temperature sensor, an atmospheric pressure sensor and a light intensity sensor in order to provide for example information to the healthcare provider on whether the drug has been properly stored to avoid possible alteration of the drug.

The information related to the drug available to the patients or to the operating status of the drug delivery device contained in the communication element is dynamically transmitted to a healthcare provider through a backscatter communication-based technology.

In this respect, the communication system 1 comprises a central unit 3, for example in the form of a hub or central unit, to be installed in the patient premises. According to the illustrated embodiment of FIG. 2, the central unit 3 comprises at least one antenna 4, a transmitter 6 configured to transmit a RF signal through the antenna 4 to a communication element 20 (FIG. 1) and a receiver 8 configured to receive a backscattered RF signal from the communication clement 20. A means for self-interference mitigation selected from the presence of at least two RF antennas at the central unit, analog carrier cancellation, digital carrier cancellation, and the use of a high pass filter, is further included to minimize interference between transmitted and received signals at the central unit 3. The central unit 3 also comprises a processing unit 10, in particular for received signal de-modulation, processing and protocol handling, and a gateway 12 for communication with the cloud services 2. Where the signal received from the communication element is modulated by a direct sequence spread spectrum modulator, the de-modulation can be a de-spreader.

The communication system 1 relies upon backscatter technology for communication from the communication element 20 to the hub 3 (the ‘uplink’), which requires a relatively simple set of hardware for the communication element 20, and drawing only limited power. This is enabled by the fact that there is no RF transmitter in the communication element since the signal is sent back to the central unit 3 using only reflection of the incident power. This has the advantage to provide a communication element that can be manufactured at minimum cost and to provide a communication system with a range large enough to cover a whole house, for example at least up to 50 m, advantageously up to at least 65 m, even more advantageously up to at least 100 m.

As illustrated in FIG. 3, the communication element 20 comprises a RF antenna 22, optionally a receiver 23 to receive data sent by the central unit (the ‘downlink’), a modulator to modulate an incident RF signal and modulate data using backscatter onto the RF signal transmitted by the central unit 3 and a processing unit 24 for interpretation of data received from the central unit, and the generation of signals to modulate back to the central unit 3. The processing unit 24 may comprise in an embodiment a direct sequence spread spectrum modulator for modulating data for transmission back to the central unit 3 as a backscattered RF signal. The communication element 20 may also comprise a power source 28, used to power the communications and processing system. The communication clement may also comprise a memory 26, configured to store sensor data measurements by different sensors as described above and provide a connection for sensors and indicators. The memory 26 may be in the form of a printed memory and/or a silicon semiconductor-based memory, for example a memory chip or an integrated memory on the communication element.

In an embodiment, the communication element 20 can be powered from the incident RF signal sent by the central unit 3 thereby providing a low energy consumption communication element. More particularly, the backscatter communication element may operate by harvesting and storing incident RF energy emitted by the central unit 3 which is rectified to generate a DC voltage to power the communication element 20. The RF signal received by the receiver 23 of the communication element 20 is processed to generate an internal clock signal that drives the internal logic within the communication element 20 in order to transmit to the central unit 3 an ID code together with the sensor data stored in the memory 26 of the communication element 20.

The communication system 1 between the central unit 3 and the communication element 20 can be achieved through an air interface protocol which uses continuous waves (CW) burst at the beginning of the transmission from the central unit 3 to simulate the powering of the communication element 20 as well as the generation of the internal clock signal within the communication element. The CW burst is followed by a command code transmitted as AM (Amplitude Modulation) on the carrier by the central unit 3. The communication element 20 processes the command and responds by load modulating the antenna by applying ASK (Amplitude Shift Keying) or PSK (Phase Shift Keying) onto the incident CW signal from the central unit 3 to generate the returned signal. In an advantageous embodiment, the communication element 20 may further comprise a Binary Amplitude Phase Shift Keying phase modulator or a Binary Phase Shift Keying phase modulator for phase modulation of the RF signal.

In an embodiment, the power source 28 is in the form of a battery, for example a printed battery. Such powered communication element using RFID (for example UHF RFID) technology removes the functionality to convert incident RF to DC (as in a conventional UHF RFID tag) and uses the power source to activate the digital functionality, and sensor systems where appropriate. This has the advantage that the range of the system is no longer limited by the need to transmit enough power to energize the communication element. Alternatively, the power source can be an energy scavenger, for example from solar energy, as a RF scavenger from sources other than the central unit 3 (hub) or in addition to the central unit 3 (hub), from heat, or from motion. In addition, the power source can comprise such energy scavenger combined with a power storage element to save up small quantities of energy for when needed.

The processing unit 10 of the central unit 3 of the communications system 1 comprises a de-modulator with a low data bandwidth rate for de-spreading the spread spectrum backscattered RF signal received from the communication element 20. The central unit 3 also comprises a low phase noise signal source 14 and a self-interference cancellation device which may comprise two antennas (not shown), i.c. first and second antenna coupled respectively to the transmitter 6 and the receiver 8 of the central unit 3. Dual antennas provide greater isolation between the signal transmitted by the central unit 3 and the backscattered RF signal emitted by the communication element 20. One or more other self-interference cancellation means can advantageously be implemented such as analog carrier cancellation, digital carrier cancellation or the use of a high pass filter.

While this invention has been shown and described as having preferred designs and features, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

1. Communication system comprising:
a. a central unit comprising at least one RF antenna, a transmitter configured to transmit a RF signal and a receiver configured to receive a backscattered RF signal; and
b. a communication element comprising a RF antenna and configured to transmit data to the central unit by backscatter of the RF signal emitted from the central unit,
wherein the communication element comprises a modulator for modulating the backscattered RF signal into a spread spectrum backscattered RF signal and the central unit comprises a means for de-spreading the spread spectrum backscattered RF signal, and wherein the communication element comprises a means for supplying power.

2. The communication system according to claim 1, wherein the transmitter of the central unit comprises a low phase noise signal source.

3. The communication system according to claim 1, wherein the central unit further comprises one or more of a two antenna configuration, a high pass filter, and carrier cancellation self-interference mitigation selected from analog and digital.

4. The communication system according to claim 1, wherein the spread spectrum backscattered RF signal is a direct sequence spread spectrum backscattered RF signal.

5. The communication system according to claim 1, wherein the communication system uses a low data bandwidth.

6. (canceled)

7. (canceled)

8. The communication system according to claim 1, wherein the operable range between the central unit and the communication element is at least 50 m, optionally at least 65 m, or optionally at least 100 m.

9. The communication system according to claim 1, wherein the communication element comprises an oscillator which is implemented as part of a conventional integrated circuit, without the use of an additional precision oscillator, for example a crystal oscillator.

10. (canceled)

11. The communication system according to claim 1, wherein the communication element further comprises a memory means.

12. The communication system according to claim 11, wherein the memory means is selected from a printed memory and a silicon semiconductor-based memory, for example a memory chip or an integrated memory on the communication element.

13. The communication system according to claim 1, wherein the communication element, or parts thereof, are fabricated using printing techniques, for example wherein the communication element comprises printed electronics.

14. The communication system according to claim 1, wherein the means for supplying power in the communication element comprises one or more of a battery, a supercapacitor and an energy harvesting means.

15. (canceled)

16. The communication system according to claim 1, wherein the communication element further comprises a modulator configured to modulate the backscatter RF signal by modulation using Binary Phase Shift Keying or Binary Amplitude Shift Keying on the reflected backscatter RF signal.

17. (canceled)

18. The communication system according to claim 1 further comprising one or more sensors, operably connected to the communication element.

19. (canceled)

20. The communication system according to claim 1, further comprising a user interface.

21. (canceled)

22. A communication element comprising:
a. a RF antenna;
b. a means configured to transmit data to the central unit by backscatter of the RF signal incident upon the antenna;
c. a means for supplying power; and
d. a modulator for modulating the backscattered RF signal to obtain a spread spectrum backscattered RF signal.

23. The communication element according to claim 22 further comprising a memory means.

24. (canceled)

25. (canceled)

26. The communication element according to claim 22, comprising printed electronics.

27. The communication element according to claim 22, wherein the means for supplying power in the communication element comprises one or more of a battery, a supercapacitor and an energy harvesting means.

28. (canceled)

29. The communication element according to claim 22, wherein the modulator is configured to modulate the backscatter RF signal by direct sequence spread spectrum modulation.

30. The communication element according to claim 22, wherein the memory means comprises a means to store and read/write data onto the communication label through a communication interface.

31. The communication element according to claim 22, further comprising one or more sensors, operably connected to the communication element.

32. (canceled)

33. (canceled)

34. The communication element according to claim 22, further comprising a user interface.

35. (canceled)

36. The communication element according to claim 22, wherein the communication element is an integral part of packaging of a consumable or a medicament.

37. A central unit comprising:
a. at least one RF antenna,
b. a transmitter configured to transmit a RF signal,
c. a receiver configured to receive a backscattered RF signal, and
d. a means for de-spreading a spread spectrum modified backscattered RF signal.

38. The central unit according to claim 37, wherein the means for de-spreading is configured to de-spread a direct sequence spread spectrum modified backscattered RF-signal.

39. The central unit according to claim 37, further comprising a means to de-modulate a Binary Phase Shift Keying or Binary Amplitude Shift Keying modulated backscattered RF signal.

40. The central unit according to claim 37, wherein the transmitter of the central unit comprises a low phase noise signal source.

41. The central unit according to claim 37, further comprising one or more of a two antenna configuration, a high pass filter, and carrier cancellation self-interference mitigation selected from analog and digital.

42. (canceled)

43. The central unit according to claim 37, wherein further comprising a modulator configured to modulate the transmitted RF signal.

44. (canceled)

45. (canceled)

46. (canceled)

47. (canceled)

48. A method of transmitting information between a central unit and a consumable or medicament container using the communication system according to claim 1.

49. (canceled)
